(19) United States
(12) Patent Application Publication
(10) Pub. No.: US 2002/0128542 A1
(43) Pub. Date: Sep. 12, 2002

(54) Physiological monitor for veterinary and human medical use and method

(76) Inventor: James E. Van Over, Colby, WI (US)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 09/803,252

(22) Filed: Mar. 9, 2001

(51) Int. Cl. A61B 5/00

(52) U.S. Cl. 600/310; 600/342

(57) ABSTRACT

An in situ apparatus for measuring a physiological or biological property of a body fluid of an animal includes a body invasive instrument carrying an optical indicator which is exposed to the body fluid and utilizing a spectrophotometric device to generate a color signal or other quantitative measurement representative of the property to be measured which is then transmitted to a receiver remote from the body and converted into a readable output. Real time measurement of rumen pH and other physiological properties in cows and other ruminants utilizes an optical indicator incorporated in a stable porous glass matrix applied to an ingestible bolus. A spectrophotometric device, microprocessor and power source generate a color signal of rumen pH or other property being measured and transmit the color signal to a receiver remote from the ruminant.

### BACKGROUND OF THE INVENTION

The present invention pertains to an apparatus and method for measuring and monitoring in situ a physiological property of a body fluid of an animal, for example, the rumen contents of a ruminant. More specifically, the apparatus and method described herein permit the measurement of a physiological property, such as rumen pH, on a continuous basis in real time in the rumens of dairy cows. However, a broad scope of monitoring the properties of bodily fluids using other common invasive apparatus is disclosed.

It is well known in veterinary and dairy sciences that rumen pH in dairy cows must be maintained within a fairly small range in order to assure good milk production and to maintain the health of the animal. Ideally, rumen pH should generally be maintained in a fairly narrow range of about 5.9 to 6.1. Temporary acid excursions down to about pH 5.5 are not unusual, but chronically low rumen pH in the range of about 5.5 to 5.0 results in subacute rumen acidosis. If ruminal pH drops below about 5.0 and remains there for any significant amount of time, acute rumen acidosis may result with often fatal consequences. Unfortunately, many of the classic clinical signs of rumen acidosis are delayed for weeks or even months after the occurrence of the low ruminal pH excursion. By the time the symptoms appear, subacute rumen acidosis will have already led to serious consequences, including rumenitis, liver abscesses, and laminitis. Other consequences of acute rumen acidosis are more severe and, even where cows survive the initial effects of acute acidosis, they may later die from resultant complications. Beyond the serious health related problems, acidosis adversely affects milk production in a very significant manner.

The ability to control and prevent rumen acidosis in cows and other ruminants could be significantly enhanced if methods and apparatus were available to monitor pH on a regular and real time basis. Unfortunately, the only presently available means for the direct rumen pH measurement in cows is to draw a sample directly from the rumen with a needle inserted through the rumen wall. Such invasive sampling cannot, of course, be done on a single cow on a regular basis, but can only be done on the basis of selective samplings from a herd on a periodic basis. Many countries outside the U.S. completely prohibit the use of needle withdrawal of rumen fluid.

Therefore, there exists a real need for the ability to measure rumen pH in real time in dairy cows and other ruminants. The ability to promptly detect and address low pH excursions can result in increased milk production and general increase in the health of the animals.

There are other physiological properties of the rumen contents of animals the measurement of which would be helpful in maintaining animal health and productivity. These include the measurement of certain anion and cation concentrations in the rumen to determine, for example, dietary cation and anion difference.

In a more general sense, there is a need and there would be great advantages in being able to monitor in situ parameters of bodily fluids in animals generally. For example, pH and other ion concentrations in blood, if they could be measured and monitored in real time, would be of substantial benefit in both human and veterinary medicine.

### SUMMARY OF THE INVENTION

In accordance with the present invention, a physiological property of a body fluid of an animal is monitored in situ and on a continuous real time basis using a body-invasive sensor and associated transmission and processing devices to generate a readable value outside the body. In accordance with a principal embodiment, a body-invasive carrier instrument is provided with an optical indicator for a property to be measured in a manner to expose the indicator to the body fluid. A light source and a photodetector are operatively connected to the indicator and a light path is provided to direct light from the light source to the indicator and from the indicator to the photodetector. The photodetector includes a photometric device to generate a color signal representative of the color of the optical indicator, and a processing device converts the color signal to a readable color value. An appropriate power source for the monitoring apparatus is also provided.

The indicator is preferably incorporated into a porous glass matrix. Where the property to be measured is pH, the optical indicator comprises a chromometric pH indicator, such as bromophenol blue, incorporated into a sol-gel glass matrix. For veterinary applications, the carrier instrument may comprise an ingestible bolus.

The carrier instrument may also comprise a catheter and, where the fluid whose property to be measured is blood, the catheter comprises an in-dwelling venous catheter. In this embodiment, the optical indicator is incorporated into a porous glass matrix that is mounted on the tip of the catheter. The light path is provided by an optical fiber that interconnects the glass matrix and an opposite catheter end outside the body. In a further embodiment, a plurality of porous glass matrices may be mounted on the catheter tip, each matrix incorporating an optical indicator for a different property of the fluid. An optical fiber interconnects each matrix to the outside of the body.

A related method of the invention includes the steps of (1) exposing a body fluid to a property-specific chromometric indicator that is incorporated into a stable porous glass matrix carried on an exposed surface of a body-invasive instrument, (2) incorporating a spectrophotometric device within the instrument operatively connected to the indicator to generate a color signal representative of the property to be measured, (3) transmitting the color signal to a receiver remote from the body, and (4) converting the signal to a readable output of the measured property.

Preferably, the spectrophotometric device includes a light source and a detector, and the method includes the additional step of transmitting light from the source to the indicator along a fiber optic path.

The basic method may also include the steps of (1) incorporating the indicator into the distal end of a catheter, and (2) exposing the fluid to the indicator by inserting the catheter into the body. The catheter preferably comprises an in-dwelling venous catheter.

In accordance with another embodiment of the present invention, rumen pH is monitored in situ and on a continuous real time basis using an ingestible sensor and associated telemetry to permit remote pH monitoring. The pH sensor technology is broadly applicable to the measurement of other physiological properties of rumen contents.

In accordance with this embodiment of the invention, a physiological monitoring device for measuring a property of the rumen contents of a ruminant comprises a housing that is ingestible by the ruminant, the housing having an enclosing wall; an optical indicator mounted in the enclosing wall and exposed to the rumen contents, the indicator being specific for a property to be measured; a light source and a photodetector mounted within the housing; a light path from the light source to the indicator and from the indicator to the detector; a spectrophotometric device operative to generate a color signal representative of the light from the indicator; a processing device operative to convert the color signal for radio transmission to a remote receiver; and, a power source for the monitoring apparatus in the housing.

The optical indicator is preferably incorporated into a porous glass matrix and in a preferred embodiment, the property to be measured is rumen pH and the optical indicator comprises a chromometric pH indicator. In this embodiment, the glass preferably comprises a sol-gel matrix and the indicator comprises bromophenol blue. In all embodiments of the invention, the housing preferably comprises a bolus which may be made from stainless steel, aluminum or thermoplastics.

Preferably, the light path comprises a light transmissive window made, for example, of glass positioned in the wall of the housing. The porous glass matrix carrying the indicator material is applied to the outside surface of the window. In a particularly preferred embodiment, the window is a semicylindrical glass layer defining an input edge for receiving light from the light source and an opposite output edge for directing light to the detector. By utilizing other physiological parameter indicators, a plurality of porous glass matrices, each incorporating an optical indicator for a different property, can be applied to the surface of the window with each property being monitored and remotely transmitted as indicated above.

In accordance with the method of the subject invention, in situ real time measurement of rumen pH comprises the steps of (1) exposing the rumen contents to a chromometric indicator incorporated into a stable porous glass matrix carried on a bolus ingestible by a ruminant; (2) providing a spectrophotometric device in the bolus which is operatively connected to the pH indicator and is operative to generate a color signal representative of rumen pH; (3) transmitting the signal to a receiver remote from the ruminant; and, (4) converting the signal to a pH value.

The method preferably includes the steps of providing the bolus with a light transmissive window, and applying the glass matrix and indicator to the outer surface of the window. The spectrophotometric device includes a light source and a detector, and the method includes the additional step of transmitting light from the source to the detector along a path defined by the window.

In accordance with a somewhat broader method of the subject invention, real time measurement of a physiological property of the rumen contents of a cow or other ruminant comprises the steps of (1) exposing the rumen contents to a property-specific chromometric indicator which is incorporated into a stable porous glass matrix, the matrix carried on the outer surface of an ingestible bolus; (2) housing a spectraphotometric device within the bolus to generate from the indicator a color signal representative of the property to be measured; (3) transmitting the color signal to a remote receiver; and (4) diverting the signal to an output of the measured property.

### BRIEF DESCRIPTION OF THE DRAWINGS

FIG. 1 is a side view of an ingestible bolus comprising the housing for the apparatus of one embodiment of the present invention.

FIG. 2 is an enlarged cross section taken on line 2-2 of FIG. 1.

FIG. 3 is an enlarged detail of a portion of FIG. 2 taken on line 3-3 thereof and additionally showing schematically other components of the system.

FIG. 4 is an enlarged detail of the light conducting wall portion of the housing carrying the optical indicator.

FIG. 5 is a generally schematic view of another embodiment of the invention incorporated into a catheter.

FIG. 6 is an enlarged sectional detail of the interconnection between the catheter tube and tip of FIG. 5.

FIG. 7 is a view of the end of the catheter tube taken on line 7-7 of FIG. 5.

FIG. 8 is a view of the end of the catheter tip taken on line 8-8 of FIG. 5.

### DETAILED DESCRIPTION OF THE INVENTION

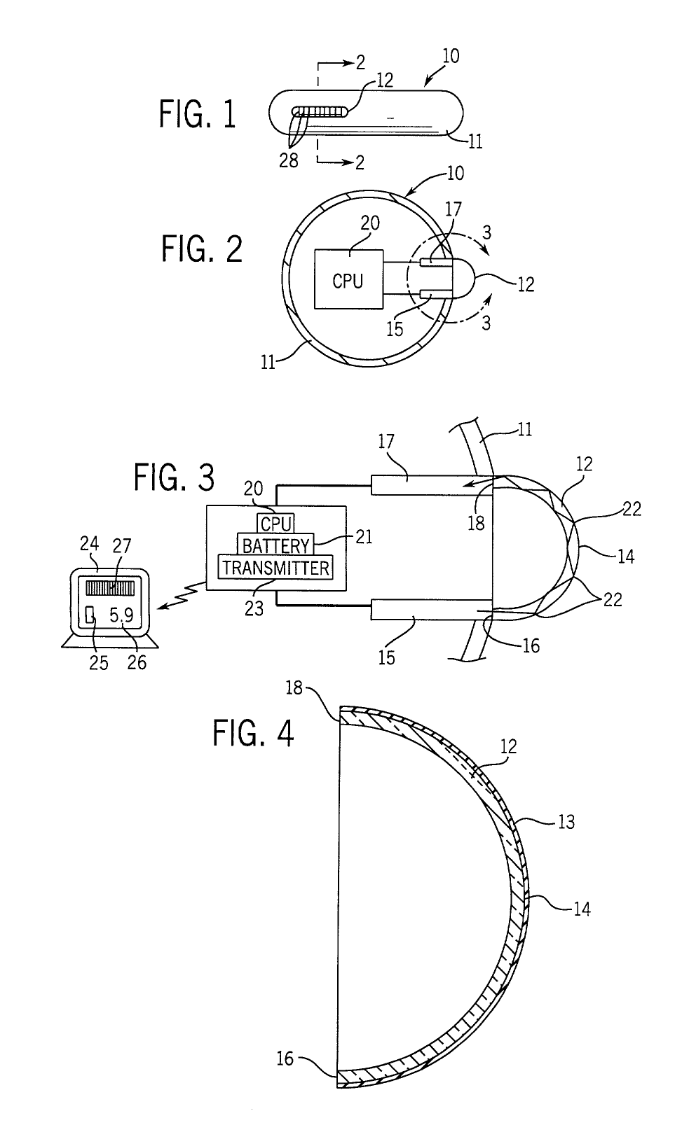

Referring to FIG. 1, an apparatus in accordance with one embodiment of the present invention is housed in a bolus 10 which, preferably, is of a size capable of being readily ingested by a dairy cow. A dairy cow can easily swallow a bolus up to 4½ inches (about 115 mm) in length and 1 inch (about 25 mm) in diameter. If necessary, however, a larger size bolus may be used if it is coated on the outside to facilitate swallowing. With the miniaturized electronic components available today, as will be described below, the size of the bolus could be readily reduced to 3¾ inches (about 95 mm) in length and ⅝ inch (about 15 mm) in diameter.

The enclosing wall 11 of the bolus 10 must be made of a material having durability and long term resistance to corrosion in the relatively harsh environment of cow's rumen, which may be compared to a large and complex fermantation vessel. Stainless steel is a preferable material for the enclosing wall 11 of the bolus, but aluminum and certain thermoplastic materials are also suitable.

The wall of the bolus 10 defines an enclosed interior and also carries a window 12 made of a light-transmitting glass or crystal material. The window is raised above the outer surface of the wall 11 and has a semi-cylindrical cross sectional shape, as shown in FIGS. 2-4. Referring particularly to FIG. 4, the outer surface of the window 12 is coated with an optical indicator material 13 that is responsive to a physiological property of the rumen contents desired to be measured. In a principal embodiment of the invention, the optical indicator comprises a pH sensor in which a suitable indicator material is suspended in a porous glass matrix 14 to provide a sensor of high stability and longevity when exposed to the contents of the rumen of a cow or other ruminant.

In particular, the optical indicator 13 is preferably fabricated in accordance with the sol-gel glass technology described in U.S. Pat. No. 5,637,507 (Wicks et al), which patent is incorporated by reference herein. For the purposes of pH monitoring and measurement, the optical indicator incorporated into the glass matrix 14 preferably comprises bromophenol blue. The sol-gel porous glass matrix 14 provides an inert and stable carrier for the pH indicator. The matrix 14 carrying the indicator may be applied to the outside of the window 12 in a thin layer, for example, 50 to 100 microns thick. The bromophenol blue indicator material, as is well known in the art, changes color with changing pH of an analyte coming in contact therewith. The color change may be detected by light passed through or reflected from the indicator matrix 14 and suitably processed to provide a color output directly indicative of pH. The output color signal may be processed to provide a direct pH value or compared visually with a color change for pH match.

Inside the enclosing bolus 10 are mounted a light source 15 (near infrared or other suitable light) positioned to direct light into an input edge 16 of the semicylindrical window 12, a photodetector 17 positioned to receive reflected light from an output edge 18 of the window 12, a microprocessor 20 for receiving and adapting a color signal from the photodetector for transmission to a receiver remote from the cow, and a power source such as a battery 21. Referring particularly to FIGS. 3 and 4, light from the light source 15 travels along the semicircular path defined by the window 12, making reflective contact with the glass matrix 14, carrying the optical indicator 13, at a number of contact points 22 along the path traveled by the light. The multi-point contact of the light with the optical indicator in the glass matrix 14 is believed to enhance the accuracy of the color signal exiting the output edge 18 and received by the photodetector 17. The color signal from the photodetector is suitably converted by the microprocessor 20 for radio transmission from a transmitter 23 within the bolus 10 to a remote receiver 24. The receiver 24 regenerates the color signal from the optical indicator 13 as an actual color display 25 and/or a numerical pH reading 26. A pH color display 25 may be visually compared with a color chart 27 by an operator at the receiver 24, the color chart based on the colors exhibited by the optical indicator over a range of pH values to which the indicator is responsive. Using bromophenol blue as the optical indicator 13, a response range of about 4 pH units, from 3.9 to 7.7 is attainable. Fully detectable and visually observable color changes are seen in pH changes as small as 0.1, thereby providing an accuracy fully appropriate for monitoring rumen pH.

The excellent stability of the indicator material in the porous sol-gel glass matrix 14 completely obviates the need for recalibration and, as a result, reliable real time pH measurements are possible over the fall life of the apparatus. With a battery 21 made with currently available technology, a battery life of several years is easily attained.

As indicated previously, optical indicators for other parameters are available or are under active development. In addition to monitoring pH, the measurement of other ion concentrations in the rumen of dairy cows is also important. Particularly for the optimization of fermentation in the rumen, the determination of dietary cation and anion difference is extremely important. This difference is determined by measuring respective sodium and potassium cations and chloride and sulfate anions. It is contemplated that optical indicators for these ions be applied to the apparatus and method of this invention as previously described for the pH indicator. Indicators for other analytes of interest in veterinary medicine already exist and others are being developed or are likely to be developed. These include indicators for volitile fatty acids (VFAs) including acetate, propionate and buterate; beta hydroxy buteric acid (BHBA), and other ketones, such as cc ketogluterate, acetoacetate and acetone. In addition, other electrolytes such as magnesium, calcium, and bicarbonate constitute important parameters whose monitoring and measurement are important in veterinary medicine. Furthermore and referring to FIG. 1, multiple indicators 28 could be applied as narrow bands to the window 12 in the bolus 10. Each band would have a different optical indicator incorporated into the porous glass matrix 14, as previously described. Each of the parameters could be measured in a similar manner on a real time basis and transmitted to a remote location for display and analysis. The determination of dietary cation and anion difference, for example, could be utilized to provide automatic control of feed formulation for each cow.

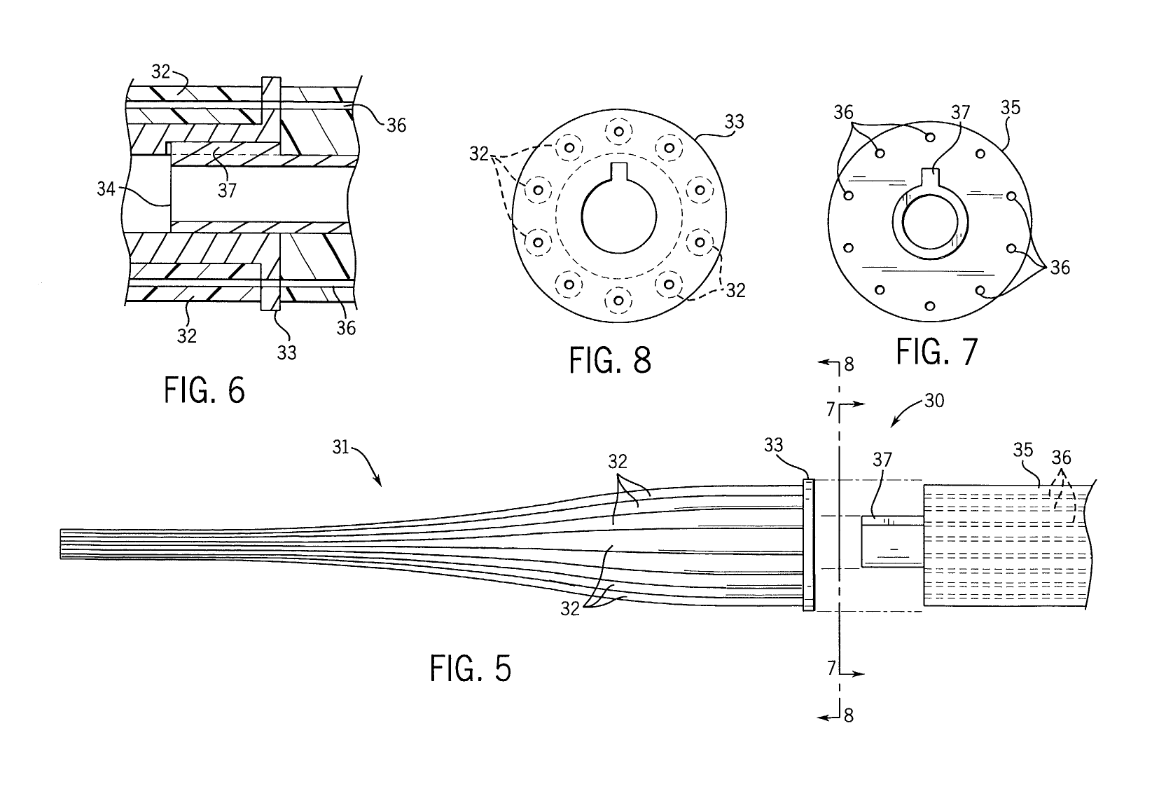

In accordance with a broader aspect of the subject invention, the indicator technology described above is applied to an apparatus useful in monitoring a physiological or biological property of other body fluids in an animal. For example, the partial pressure of carbon dioxide (pCO₂) and oxygen (pO₂) in the blood are important parameters, the measurement of which can indicate much about the metabolic and respiratory condition of an animal or a patient. Referring also to FIGS. 5-8, the glass matrix indicator technology is shown applied to an in-dwelling catheter 30 that could be used in either human or veterinary medicine applications. A catheter tip 31 (shown separated from the catheter tube 35) comprises a plurality of glass matrix tails 32, each of which incorporates an optical indicator specially selected to be responsive to a different measured property or parameter. The indicator tails 32 are distributed around and attached to an annular connector 33 adapted to receive a male connector 34 on the end of the intravenous tube 35 leading from the patient. Preferably, the connector 34 comprises the end of the catheter through which fluids are administered to the tip as in a conventional catheter. The outer wall of the IV tube includes a plurality of fiber optic transmission lines 36 responding to the indicator tails 32 on the tip of the catheter. The connector pieces 33 and 34 are provided with an appropriate alignment device, such as a pilot rib 37, to assure appropriate alignment between each indicator tail 32 and its respective dedicated fiber optic line 36. The fiber optic lines 36 are extremely small for incorporation into an IV tube 35 of conventional diameter, the fiber optic lines being of a diameter, for example, of only 150 microns.

The light source and photodetector, as well as the microprocessor and power source for generating the necessary color signals are all located outside the body of the animal or patient for appropriate connection to the end of the catheter tube 35. It is contemplated that each indicator would utilize two optically connected indicator tails 32 and a corresponding pair of fiber optic lines 36. Light from the external source would be transmitted to the tip via one fiber optic line and returned for processing into a color signal via the other fiber optic line of the pair. The actual color display of the parameter or parameters being monitored may be displayed as shown in FIG. 3 and previously described with respect to that embodiment. Thus, an appropriate receiver 24 including a CRT color display 25 and/or a direct pH reading 26, with or without a color chart 27, may be utilized.

In addition to the previously described embodiment incorporating the indicator technology into an intravenous catheter, the invention is also applicable to a urinary catheter or other type of invasive instrument which may be inserted into a body cavity for direct contact with a body fluid. For example, a urinary catheter incorporating the detector technology of the subject invention could be utilized to monitor sugar levels in diabetics or to monitor renal function and failure based on detectable biological parameters.

It is also contemplated that indicators utilizing surface plasmon resonance could also be used with any of the embodiments of the invention described herein. This technology permits the remote quantitative determination of molecules adsorbed on an exposed probe tip. As in the previously described embodiments, quantitative measurements utilize reflected light intensity and the same type of fiber optic carriers of the incident and reflected light signals.

I claim:

1. A physiological monitoring apparatus for measurement of a property of a body fluid of an animal:
a body-invasive carrier instrument;
an optical indicator for a property to be measured mounted on the carrier instrument and exposed to the body fluid;
a light source and a photodetector operatively connected to the indicator;
a light path to direct light from the light source to the indicator and from the indicator to the photodetector;
the photodetector including a photometric device capable of generating a color signal representative of the color of the optical indicator;
a processing device capable of converting the color signal to a readable color value; and,
a power source for the monitoring apparatus.

2. The apparatus as set forth in claim 1 wherein the optical indicator is incorporated into a porous glass matrix.

3. The apparatus as set forth in claim 2 wherein the property to be measured is pH and the optical indicator comprises a chromometric pH indicator.

4. The apparatus as set forth in claim 3 wherein the glass comprises a sol-gel matrix and the indicator comprises bromophenol blue.

5. The apparatus as set forth in claim 1 wherein the carrier instrument comprises a bolus.

6. The apparatus as claimed in claim 1 wherein the carrier instrument comprises a catheter.

7. The apparatus as set forth in claim 6 wherein the fluid is blood and the catheter comprises a venous catheter.

8. The apparatus as set forth in claim 6 wherein the fluid is urine and the catheter comprises a urinary catheter.

9. The apparatus as set forth in claim 6 wherein the optical indicator is incorporated into a porous glass matrix and said matrix is mounted on the catheter tip.

10. The apparatus as set forth in claim 9 wherein the light path comprises an optical fiber interconnecting the glass matrix and an opposite catheter end outside the body.

11. The apparatus as set forth in claim 9 wherein a plurality of porous glass matrices are mounted on the catheter tip, each matrix incorporating an optical indicator for a different property, and an optical fiber for each matrix.

12. A method for real time measurement of a physiological property of a body fluid of an animal comprising the steps of:
(1) exposing the fluid to a property-specific chromometric indicator incorporated into a stable porous glass matrix carried on an exposed surface of a body-invasive instrument;
(2) incorporating a spectrophotometric device within the instrument operatively connected to the indicator to generate a color signal representative of the property to be measured;
(3) transmitting the color signal to a receiver remote from the body; and,
(4) converting the signal to a readable output of the measured property.

13. The method as set forth in claim 12 wherein the spectrophotometric device includes a light source and a detector, and including the step of transmitting light from the source to the indicator along a path defined by the window.

14. The method as set forth in claim 12 including steps of:
(1) incorporating the indicator into a distal end of a catheter; and,
(2) exposing the fluid to the indicator by inserting the catheter into the body.

15. The method as set forth in claim 14 wherein the catheter comprises an in-dwelling venous catheter.

16. The method as set forth in claim 15 wherein the catheter comprises a urinary catheter.

17. A physiological monitoring apparatus for measurement of a property of the rumen contents of a ruminant comprising:
a ruminant ingestible housing having an enclosing wall;
an optical indicator for a property to be measured mounted in the enclosing wall and exposed to the rumen contents;
a light source and a photodetector within the housing;
a light path in the housing directing light from the light source to the indicator and from the indicator to the detector;
the detector including a photometric device capable of generating a color signal representative of the color of the optical indicator;
a processing device in the housing capable of converting the color signal for radio transmission to a receiver remote from the ruminant; and,
a power source for the monitoring apparatus in the housing.

18. The apparatus as set forth in claim 17 wherein the optical indicator is incorporated into a porous glass matrix.

19. The apparatus as set forth in claim 18 wherein the property to be measured is pH and the optical indicator comprises a chromometric pH indicator.

20. The apparatus as set forth in claim 19 wherein the glass comprises a sol-gel matrix and the indicator comprises bromophenol blue.

21. The apparatus as set forth in claim 17 wherein the housing comprises a bolus.

22. The apparatus as set forth in claim 21 wherein the bolus is made of a material selected from the group consisting of stainless steel, aluminum and thermoplastics.

23. The apparatus as set forth in claim 18 wherein the light path comprises a light transmissive window in the wall of the housing, and the porous glass matrix is applied to the outside surface of the window.

24. The apparatus as set forth in claim 23 wherein a plurality of porous glass matrices are applied to the surface of the window, each matrix incorporating an optical indicator for a different property.

25. A method for in situ real time measurement of pH in the rumen of a ruminant animal comprising the steps of:
(1) exposing the rumen contents to a chromometric pH indicator incorporated into a stable porous glass matrix carried on a ruminant ingestible bolus;
(2) providing in the bolus a spectrophotometric device operatively connected to the indicator and operative to generate a color signal representative of rumen pH;
(3) transmitting the color signal to a receiver remote from the ruminant animal; and,
(4) converting the signal to a pH value.

26. The method as set forth in claim 25 including the steps of:
(1) providing the bolus with a light transmissive window; and,
(2) applying the glass matrix and indicator to the outer surface of the window.

27. A method for real time measurement of a physiological property of the rumen contents of a ruminant comprising the steps of:
(1) exposing the rumen contents to a property-specific chromometric indicator incorporated into a stable porous glass matrix carried on the outer surface of an ingestible bolus;
(2) incorporating a spectrophotometric device within the bolus operatively connected to the indicator to generate a color signal representative of the property to be measured;
(3) transmitting the color signal to a receiver remote from the ruminant; and,
(4) converting the signal to an output of the measured property.

28. An apparatus for continuous real time measurement of a physiological property of a body fluid of an animal:
a body-invasive carrier instrument comprising:
an optically sensitive indicator device for a property to be measured mounted on the carrier instrument and exposed to the body fluid;
a light source and a photodetector device operatively connected to the indicator device, including a light path to direct light from the light source to the indicator device and from the indicator device to the photodetector;
the photodetector device including a processor capable of generating a signal representative of the quantitative measurement; and
a processing device capable of converting the signal to a readable values.
